(19) United States
(12) Patent Application Publication
(10) Pub. No.: US 2018/0315490 A1
(43) Pub. Date: Nov. 1, 2018

(54) METHOD FOR ASSESSING RESPONSE VALIDITY WHEN USING DICHOTOMOUS ITEMS WITH HIGH GRANULARITY

(71) Applicant: Mark Ellis Jaruzel, II, Farmington Hills, MI (US)

(72) Inventor: Mark Ellis Jaruzel, II, Farmington Hills, MI (US)

(73) Assignee: The Live Network, Inc., Farmington, MI (US)

(21) Appl. No.: 15/964,065

(22) Filed: Apr. 26, 2018

Related U.S. Application Data
(60) Provisional application No. 62/490,123, filed on Apr. 26, 2017.

(51) Int. Cl. G16H 10/20; G16H 50/70; G16H 50/30; G06N 3/08; G06F 3/0484; G16H 10/60

(52) U.S. Cl. CPC G16H 10/20; G16H 50/70; G16H 10/60; G06N 3/08; G06F 3/04847; G16H 50/30

(57) ABSTRACT

The present disclosure relates to a method for assessing how a particular patient's responses may vary from those that are typically given on an item or instrument that relies on high granularity, dichotomous, responses. This method allows for the quantifiable description of patterns of possible overreporting, underreporting, “yeasaying,” “naysaying,” favoring one half of a representative continuum in a manner unrelated to item content, attempting to portray oneself in an overly favorable light, or attempting to portray oneself in an overly negative light. This method allows for validity checks without recourse to generating additional, specific, validity check items possibly modifying the times required to construct and train various machine learning (ML) systems including artificial neural networks (ANNs).

### REFERENCE TO RELATED APPLICATIONS

This application claims an invention which was disclosed in Provisional Application No. 62/490,123, filed Apr. 26, 2017, entitled “METHOD FOR ASSESSING RESPONSE VALIDITY WHEN USING DICHOTOMOUS ITEMS WITH HIGH GRANULARITY”. The benefit under 35 USC § 119(e) of the United States provisional application is hereby claimed, and the aforementioned application is hereby incorporated herein by reference.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The invention pertains to the fields of healthcare services, psychodiagnostic assessment, and machine learning. More particularly, the invention pertains to a computer-implemented system for and method of assessing the relative and normative validity of responses on items, scales, and/or instruments that employ dichotomous items with high granularity and a continuous set of possible responses.

### Description of Related Art

A mental health disorder, also commonly referred to as a mental illness, is a pattern of mood, cognition, behavior, or personality that occurs in a person and is thought to cause distress or disability that is not a normal part of development or culture. Mental health disorders are quite common. In the United States, the American Psychiatric Association estimates that over 68 million Americans will meet diagnostic criteria for a psychiatric or substance use disorder in a given year. Studies in several English-speaking countries have suggested that over the course of the lifespan it is more common to meet the criteria for a mental health disorder than to not meet criteria for a mental health disorder. The costs associated with treated, undertreated, and untreated mental illnesses are extremely high with The World Economic Forum estimating that worldwide costs were $2.5 trillion for the year 2010.

Access to adequate assessment and care for mental health disorders is lacking in many parts of the United States. The Substance Abuse and Mental Health Services Administration (SAMHSA) estimates that fewer than 50% of adults meeting diagnostic criteria for a mental health disorder receive any treatment for that disorder. The combination of stigma, low provider density areas, and inadequate treatment resources presently complicates the practice of mental healthcare. One component that will contribute to more adequately addressing mental health concerns will be increased identification of existing concerns through quick and accurate diagnostic tools.

One commonly employed method of psychodiagnostics is to ask patients questions utilizing multiple choice questions. These questions can be simple, forced-choice, dichotomous items (i.e. True or False questions) or they can be several point items often called Likert-style items (i.e. Strongly Disagree, Disagree, Neutral, Agree, Strongly Agree). These kinds of 2 to 7 point items initially emerged prior to the advent of modern computers and were a part of early psychodiagnostic instruments that were typically hand scored such as the initial Minnesota Multiphasic Personality Inventory (MMPI).

The move from forced choice items where the respondent was only offered two choices to items where the respondent was offered 5 or 7 items offered a range of advantages in the assessment of opinions, behavior, traits, and distress. It allowed researchers and clinicians to mitigate the effects of social desirability bias because respondents might be more willing to endorse a response such as “Somewhat Agree” on a 5 or 7-point item than they might be to endorse an “Agree” response on a 2-point item. Likert-style items offered a wider range of possible scores in a comparable sized scale than would be possible with a 2-item scale (i.e. a scale comprised of 4 2-point items might have a total score range of 4, while a scale comprised of 4 5-point items might have a total score range of 16). Likert-style items also allowed greater power and flexibility in the statistical tests used to analyze the results offered.

While the move from 2-point items to Likert-style items offered several advantages, it was not without its drawbacks. One of the key drawbacks is that the quantitative interpretation of the results of scores from Likert-style items is problematic. Simple parametric tests are likely inadequate because it is not clear that there is an equal change in respondent-perceived value between each item on a multipoint scale (i.e. the move from “Neutral” to “Somewhat Agree” is not the same as the move from “Somewhat Agree” to “Strongly Agree”). Because the scales are not comprised of continuous data with a fixed interval, the statistical tests germane to working with interval and ratio data are not strictly speaking applicable. This has led to confusion in interpretation and the potential loss of descriptive power in statistical and clinical analyses.

One response to some of the drawbacks inherent in 2-point items and Likert-style items, has been the recent move to continuous items with high granularity being used in the assessment of psychological distress and psychological traits. These items offer solutions to some of the concerns related to statistical inference and the development of machine learning (ML) test sets and ML products.

That said, traditional 2-point items and Likert-style items have benefited from established methods for the detection and quantification of patterns of nonresponsiveness, bias, and active deception in administered protocols. To date, there has not been a set of procedures described that demonstrate how to perform similar forms of detection and quantification for continuous items with high granularity.

The typical threats to protocol validity in 2-point scales and Likert-style scales have been overreporting, underreporting, “yeasaying,” and “naysaying.” Overreporting occurs when a respondent intentionally or unintentionally exaggerates the presence of a particular trait or symptom. Underreporting occurs when a respondent intentionally or unintentionally minimizes the presence of a particular trait or symptom. “Yeasaying” is a pattern whereby a respondent demonstrates a bias towards over agreement with item statements (i.e. when in doubt agree). “Naysaying” is a pattern whereby a respondent demonstrates a bias towards over disagreement with item statements (i.e. when in doubt disagree).

### SUMMARY OF THE INVENTION

The present disclosure relates to a method for assessing how a particular patient's responses may vary from those that are typically given on an item or instrument that relies on high granularity, dichotomous, responses. This method allows for the quantifiable description of patterns of possible overreporting, underreporting, “yeasaying,” “naysaying,” favoring one half of a representative continuum in a manner unrelated to item content, attempting to portray oneself in an overly favorable light, or attempting to portray oneself in an overly negative light. This method allows for validity checks without recourse to generating additional specific validity check items which might be easy for a respondent to identify and subvert.

The invention is a method amenable to implementation in computer software, hardware, or a combinational instance thereof, that allows for the assessment of potential validity concerns on items, scales, and/or instruments that employ dichotomous items with high granularity. Traditional methods of assessing protocol validity are better suited to True or False or Yes or No items that are binary or typically involve 7 or fewer data points (as in a Likert Scale). The present disclosure offers a method to quantify possible problems with validity for high granularity, continuous, items that could easily involve 1,000 or more possible points within the range of allowable responses.

By looking at a patient's pattern of midpoint clustering versus clustering at the extremes on a continuous item and comparing that pattern with a set of norms for midpoint clustering versus clustering at the extremes, a ML system can be trained to offer a prediction about a patient's potential tendency towards generally underreporting or overreporting on an item, category, or instrument. Additionally, the potential validity or lack of validity of the protocol can be estimated by this method.

By looking at a patient's pattern of answering in the affirmative or avoiding answering in the affirmative on item content and comparing that pattern with a set of norms for answering in the affirmative or avoiding answering in the affirmative, a ML system can be trained to offer a prediction about a patient's potential tendency towards a biased pattern of responding on a scale or instrument. Additionally, the potential validity or lack of validity of the protocol can be estimated by this method.

By looking at a patient's pattern of clustering at one particular end of a dichotomous continuum and comparing that pattern with a set of norms for answering in the extreme, a ML system can be trained to offer a prediction about a patient's potential tendency towards a biased pattern of responding and the potential validity or lack of validity of the protocol returned.

The present invention is not intended to be limiting in the nature of the entity that is the patient. It is expected that the present invention will be used by a diverse range of healthcare professionals and patients.

### BRIEF DESCRIPTION OF THE DRAWINGS

Further advantageous features of the present invention will become more apparent when the following detailed description is taken along with reference to the accompanying drawings in which:

FIG. 1 shows a pattern of bias towards the left side of a series of continuous, dichotomous, items.

FIG. 2 shows a pattern of bias towards the right side of a series of continuous, dichotomous, items.

FIG. 3 shows an example of atypical clustering at the middle of a series of continuous, dichotomous, items.

FIG. 4 shows an example of atypical clustering at the extremes of a series of continuous, dichotomous items.

### DETAILED DESCRIPTION OF THE INVENTION

As discussed above, the present invention provides systems and methods that allow for assessing how a particular patient's responses may vary from those that are typically given on an item or instrument that relies on high granularity, dichotomous, responses. This method allows for the quantifiable description of patterns of possible overreporting, underreporting, “yeasaying,” “naysaying,” favoring one half of a representative continuum in a manner unrelated to item content, attempting to portray oneself in an overly favorable light, or attempting to portray oneself in an overly negative light. This method allows for validity checks without recourse to generating additional specific validity check items which might be easy for a respondent to identify and subvert.

Those sufficiently skilled in the arts involved will also readily recognize that the design elements in various embodiments of the present invention could be highly varied and yet still achieve functional equivalency.

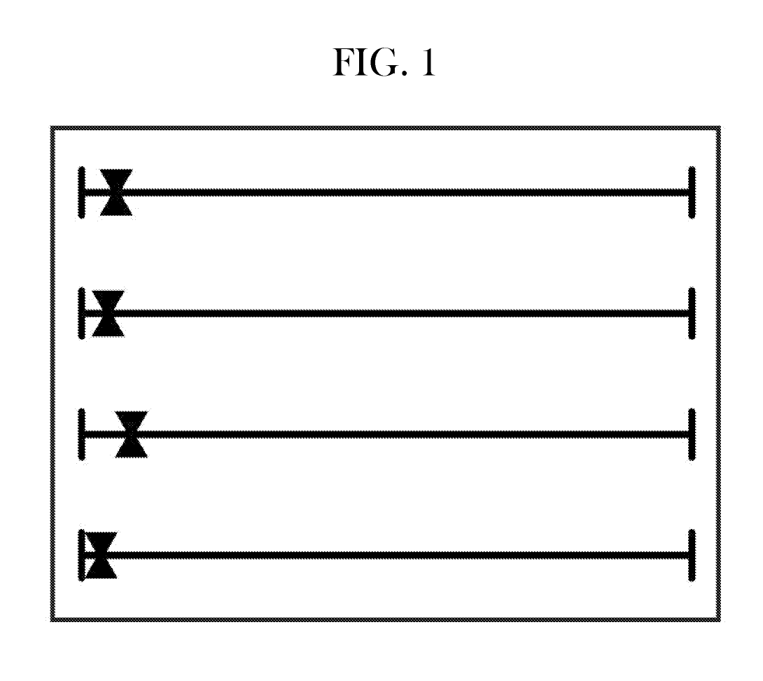

FIG. 1 illustrates a hypothetical patient that has demonstrated an atypical pattern of responding to the left of the midpoint on a set of dichotomous items with high granularity. In an ideal assessment scenario, similarly themed items will be constructed and arranged so that items related to a particular construct will be scored both right to left and left to right. In this way, a consistent pattern of left responding would imply a test-taking behavior that is less related to item content and may represent a form of content nonresponding that poses a threat to protocol validity.

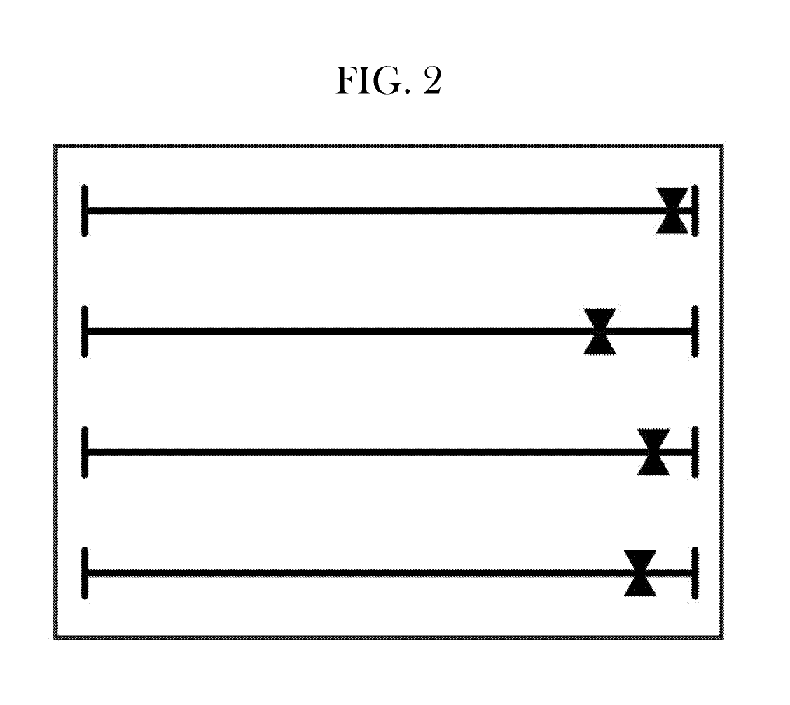

FIG. 2 illustrates a hypothetical patient that has demonstrated an atypical pattern of responding to the right of the midpoint on a set of dichotomous items with high granularity. In an ideal assessment scenario, similarly themed items will be constructed and arranged so that items related to a particular construct will be scored both right to left and left to right. In this way, a consistent pattern of right responding would imply a test-taking behavior that is less related to item content and may represent a form of content nonresponding that poses a threat to protocol validity.

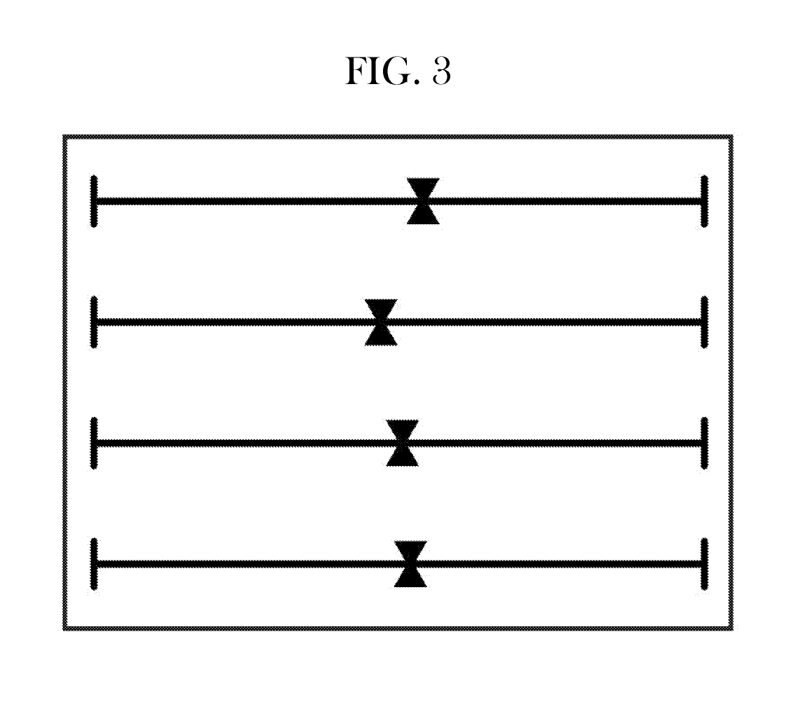

FIG. 3 illustrates a hypothetical patient that has demonstrated an atypical pattern of responding by clustering at the midpoint on a set of dichotomous items with high granularity. In an ideal assessment scenario, several items in an instrument will be constructed and arranged so that most respondents are likely to answer in the extreme and the atypical response is actually a mid-range response. In this way, a consistent pattern of middle responding would imply a test-taking behavior that is less related to item content and may represent a form of content nonresponding that poses a threat to protocol validity.

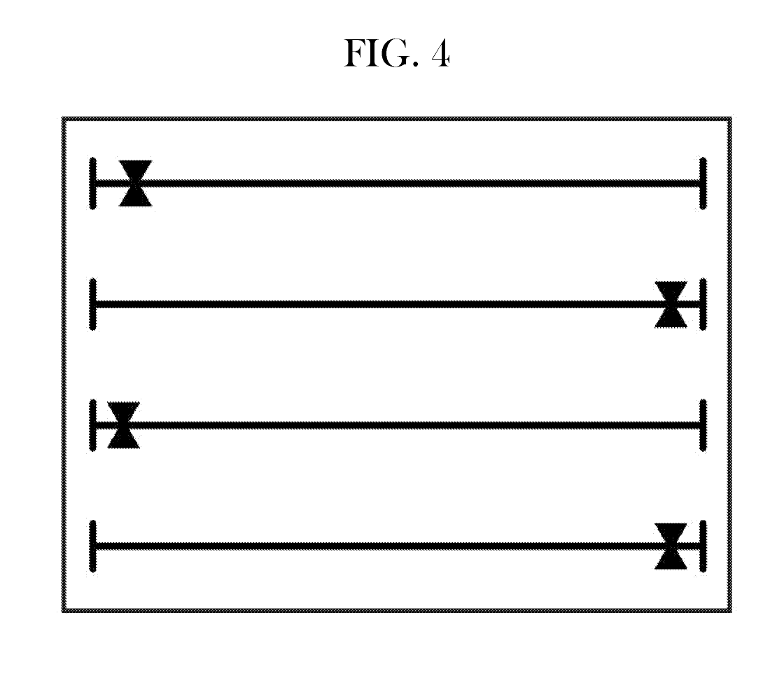

FIG. 4 illustrates a hypothetical patient that has demonstrated an atypical pattern of responding by clustering at the extremes on a set of dichotomous items with high granularity. In an ideal assessment scenario, several items in an instrument will be constructed and arranged so that most respondents are likely to answer in the middle and the atypical response is any non-mid-range response. In this way, a consistent pattern of extreme responding would imply a test-taking behavior that is less related to item content and may represent a form of content nonresponding that poses a threat to protocol validity.

Various user interfaces and embodiments were described above in some detail with reference to the drawings, wherein like reference numerals represented like parts and assemblies throughout the several views. Any of the preceding references to the various embodiments does not limit the scope of the claims attached hereto. Additionally, any examples set forth in this specification are not intended to be limiting and merely set forth some of the many possible embodiments for the appended claims. It is understood that various omissions and substitutions of equivalents are contemplated as circumstances may suggest or render expedient, but these are intended to cover applications or embodiments without departing from the spirit or scope of the claims attached hereto. Also, it is to be understood that any of the phraseology and terminology that were used herein were for the purpose of description and should not be regarded as limiting.

Any of the devices/servers/CPUs in the above-described systems may include a bus or other communication mechanism for communicating information, which interconnects subsystems and devices, such as processor, system memory (e.g., RAM), static storage device (e.g., ROM), disk drive (e.g., magnetic or optical), communication interface (e.g., modem or Ethernet card), display (e.g., CRT or LCD), input device (e.g., keyboard, touchscreen). The system component performs specific operations by the processor executing one or more sequences of one or more instructions contained in system memory. Such instructions may be read into system memory from another computer readable/usable medium, such as static storage device or disk drive. In alternative embodiments, hard-wired circuitry may be used in place of or in combination with software instructions to implement the invention. Thus, embodiments of the invention are not limited to any specific combination of hardware circuitry and/or software.

Any use of the word “screen,” “display,” or reference to a like concept above should be taken to mean a range of interfaces including but not limited to: a computer screen, a smartphone screen, a tablet screen, or an augmented reality screen or similar interface where a physical screen is lacking. Any references to a screen anywhere above are for the sake of brevity and should not be construed as a limitation on the types of devices or interfaces that can be utilized in various embodiments of this invention.

In an embodiment of the invention, execution of the sequences of instructions to practice the invention is performed by a single computing system. According to other embodiments of the invention, two or more computing systems coupled by a communication link (e.g., LAN, PTSN, or wireless network) may perform the sequence of instructions required to practice the invention in coordination with one another. The system component may transmit and receive messages, data, and instructions, including program, i.e., application code, through communication link and communication interface. Received program code may be executed by the processor as it is received, and/or stored in disk drive, or other non-volatile storage for later execution.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Methods recited herein may be carried out in any order of the recited events which is logically possible, as well as in the recited order of events.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

Accordingly, it is to be understood that the embodiments of the invention herein described are merely illustrative of the application of the principles of the invention. The breadth of the present invention is not to be limited to the examples provided, illustrated embodiments and/or the subject specification, but rather only by the scope of claim language associated with this disclosure.

What is claimed is:

1. An integrated computer-implemented system for assessing the profile validity and reliability of patient-communicated attitudes, values, opinions, traits, indicators of health, and symptoms of distress, facilitating improved patient assessment when using dichotomous items with high granularity comprising:
A database configured to store application data (all internal and external programs required to run the system) and patient response data;
A plurality of dichotomous items and categories related to attitudes, values, opinions, traits, indicators of health, and symptoms of distress;
A set of norms for the dichotomous items and categories;
A set of norms for patient patterns of midpoint clustering versus clustering at the extremes;
A set of norms for patient patterns of answering in the affirmative or avoiding answering in the affirmative;
A set of norms for patient clustering at one particular end of a dichotomous continuum;
A display configured to receive patient input;
A display configured to display a graphical user interface that includes a moveable element with high granularity that presents dichotomous choices allowing a patient or other person to move the element across the available range of values via means of touch, gesture, computer mouse dragging, or similar interactions with the interface;
A computer-implemented processor configured to use artificial intelligence to analyze patterns, to transmit data to and receive data from patients and healthcare professionals across a range of devices and interfaces (including but not limited to: laptop computers, tablets, smartphones, mobile devices, augmented reality displays, wearables, and smart devices), and to transmit data to and receive data from a database;
A computer algorithm that compares patient provided scores on dichotomous items and categories to the existing norms on those items and categories;
A second computer algorithm that specifically looks at a patient's pattern of midpoint clustering versus clustering at the extremes and compares that pattern with the existing norms for midpoint clustering versus clustering at the extremes in order to offer a prediction about a patient's potential tendency towards generally underreporting or overreporting on an item, category, or instrument and the potential validity or lack of validity of the protocol returned;
A third computer algorithm that specifically looks at a patient's pattern of answering in the affirmative or avoiding answering in the affirmative on item content and compares that pattern with existing norms for answering in the affirmative or avoiding answering in the affirmative in order to offer a prediction about a patient's potential tendency towards a biased pattern of responding and the potential validity or lack of validity of the protocol returned;
A fourth computer algorithm that specifically looks at patient's pattern of clustering at one particular end of a dichotomous continuum and compares that pattern with existing norms for answering in the extreme in that way in order to offer a prediction about the patient's potential tendency towards a biased pattern of responding and the potential validity or lack of validity of the protocol returned;
A fifth computer algorithm that specifically looks at a patient's pattern of giving potentially biased responses of the kinds described above and compares that pattern with existing norms in order to offer an overall assessment of the patient's potential tendency towards a biased pattern of responding and the potential validity or lack of validity of the protocol returned.

2. The system of claim 1, wherein the patient is a person or other entity seeking professional consultation, education, assessment, diagnosis, intervention, or treatment.

3. The system of claim 1, wherein the database has been secured through encryption.

4. The system of claim 1, wherein the computer-implemented processor has been configured to use artificial intelligence (including but not limited to: deep learning, neural network modeling, parallel distributed processing, low-rank matrix factorization, regression analysis, vectorization, and skip thought vectors) to analyze patterns in patient data to initially suggest diagnosis and prognosis as well as advantageous and disadvantageous prescribed interventions for an individual patient.

5. The system of claim 1, wherein the patient interaction with the graphical user display elements can occur before, during, and/or after the rendering of professional services so serve such purposes as: initial assessment, cumulative assessment, summative assessment, diagnosis, feedback, prognosis, risk assessment, service or treatment matching, intervention matching, and provider matching.

6. The system of claim 1, wherein the plurality of dichotomous items consists of less than forty pairs of items.

7. A computer program product for use in conjunction with a computer device of the type having a processor and a screen, the computer program product comprising a computer readable, non-transitory, storage medium and instructions thereon (or a combinational equivalent of software and hardware whether embodied in a single device or a range of networked devices that is functionally equivalent) for assessing the profile validity and reliability of patient-communicated attitudes, values, opinions, traits, indicators of health, and symptoms of distress, facilitating improved patient assessment when using dichotomous items with high granularity comprising the steps of:
Identifying a plurality of dichotomous items and categories consisting of attitudes, values, opinions, traits, and indicators of health and symptoms distress that are of relevance to clinical assessment, diagnosis, treatment, and prognosis;
Identifying a plurality of scores for various items and collections of items that serve to predict clinically relevant phenomena that are of relevance to clinical assessment, diagnosis, treatment, and prognosis;
Identifying norms for patient patterns of midpoint clustering versus clustering at the extremes;
Identifying norms for patient patterns of answering in the affirmative or avoiding answering in the affirmative;
Identifying norms for patient patterns of clustering at one particular end of a dichotomous continuum;
The presentation to the patient, via a screen or other graphical user interface, of a range of dichotomous choices allowing a patient or other person to move the element across the available range of values via means of touch, gesture, computer mouse dragging, or similar interactions with the interface;
The tabulation of scores generated via the range of values expressed by the patient or other person via the interface;
The execution of computer algorithm that compares patient provided scores on dichotomous items and categories to the existing norms on those items and categories;
The execution of a second computer algorithm that specifically looks at a patient's pattern of midpoint clustering versus clustering at the extremes and compares that pattern with the existing norms for midpoint clustering versus clustering at the extremes in order to offer a prediction about a patient's potential tendency towards generally underreporting or overreporting on an item, category, or instrument and the potential validity or lack of validity of the protocol returned;
The execution of a third computer algorithm that specifically looks at a patient's pattern of answering in the affirmative or avoiding answering in the affirmative on item content and compares that pattern with existing norms for answering in the affirmative or avoiding answering in the affirmative in order to offer a prediction about a patient's potential tendency towards a biased pattern of responding and the potential validity or lack of validity of the protocol returned;
The execution of fourth computer algorithm that specifically looks at patient's pattern of clustering at one particular end of a dichotomous continuum and compares that pattern with existing norms for answering in the extreme in that way in order to offer a prediction about the patient's potential tendency towards a biased pattern of responding and the potential validity or lack of validity of the protocol returned;
The execution of a fifth computer algorithm that specifically looks at a patient's pattern of giving potentially biased responses of the kinds described above and compares that pattern with existing norms in order to offer an overall assessment of the patient's potential tendency towards a biased pattern of responding and the potential validity or lack of validity of the protocol returned;
The presentation of the scores and/or predictions to a patient and/or professional or other entity.

8. The computer program product of claim 7, wherein the plurality of dichotomous items consists of less than forty pairs of items.

9. The computer program product of claim 7, wherein the plurality of dichotomous items consists forty or more pairs of items.

10. A method for assessing the profile validity and reliability of patient-communicated attitudes, values, opinions, traits, indicators of health, and symptoms of distress, facilitating improved patient assessment when using dichotomous items with high granularity comprising the steps of:
Identifying a plurality of dichotomous items and categories consisting of attitudes, values, opinions, traits, and indicators of health and symptoms of distress that are of relevance to clinical assessment, diagnosis, treatment, and prognosis;
Identifying a plurality of scores for various items and collections of items that serve to predict clinically relevant phenomena that are of relevance to clinical assessment, diagnosis, treatment, and prognosis;
Identifying norms for patient patterns of midpoint clustering versus clustering at the extremes;
Identifying norms for patient patterns of answering in the affirmative or avoiding answering in the affirmative on item content;
Identifying norms for patient patterns of clustering at one particular end of a dichotomous continuum;
Tabulating scores generated via the range of values expressed by the patient or other person in response to the dichotomous items and categories;
Performing a statistical comparison of the patient-provided scores on dichotomous items and categories with the existing norms on those items and categories;
Performing a second statistical comparison of a patient's pattern of midpoint clustering versus clustering at the extremes with the existing norms for midpoint clustering versus clustering at the extremes in order to offer a prediction about a patient's potential tendency towards generally underreporting or overreporting on an item, category, or instrument and the potential validity or lack of validity of the protocol returned;
Performing a third statistical comparison of a patient's pattern of answering in the affirmative or avoiding answering in the affirmative on item content and comparing that pattern with existing norms for answering in the affirmative or avoiding answering in the affirmative in order to offer a prediction about a patient's potential tendency towards a biased pattern of responding and the potential validity or lack of validity of the protocol returned;
Performing a fourth statistical comparison of a patient's pattern of clustering at one particular end of a dichotomous continuum and comparing that pattern with existing norms for answering in the extreme in that way in order to offer a prediction about the patient's potential tendency towards a biased pattern of responding and the potential validity or lack of validity of the protocol returned;
Performing a fifth statistical comparison of a patient's pattern of giving potentially biased responses of the kinds described above with existing norms in order to offer an overall assessment of the patient's potential tendency towards a biased pattern of responding and the potential validity or lack of validity of the protocol returned;
Presenting the scores and/or predictions to a patient and/or professional or other entity.

11. The method of claim 10 wherein the statistical comparison of a patient's pattern of midpoint clustering versus clustering at the extremes with the existing norms for midpoint clustering versus clustering at the extremes is accomplished by measuring the patient's mean distance from each of their scores to the central value in the range of possible values.

12. The method of claim 10 wherein the statistical comparison of a patient's pattern of midpoint clustering versus clustering at the extremes with the existing norms for midpoint clustering versus clustering at the extremes is accomplished by measuring the mean distance from each of their scores to the mean values returned on the items by the total sample norms.

13. The method of claim 10 wherein the statistical comparison of a patient's pattern of answering in the affirmative or avoiding answering in the affirmative on item content with existing norms for answering in the affirmative or avoiding answering in the affirmative is accomplished by comparing their scores to the established T-scores for each item.

14. The method of claim 10 wherein the statistical comparison of a patient's pattern of answering in the affirmative or avoiding answering in the affirmative on item content with existing norms for answering in the affirmative or avoiding answering in the affirmative is accomplished by comparing each of their scores to the mean values returned on the items by the total sample norms.

15. The method of claim 10 wherein the statistical comparison of a patient's pattern of clustering at one particular end of a dichotomous continuum and compared with existing norms for answering in the extreme is accomplished by comparing their scores to the established T-scores for each item.

16. The method of claim 10 wherein the statistical comparison of a patient's pattern of clustering at one particular end of a dichotomous continuum and compared with existing norms for answering in the extreme is accomplished by comparing each of their scores to the mean values returned on the items by the total sample norms.

17. The method of claim 10 wherein the statistical comparison of a patient's pattern of giving potentially biased responses of the kinds described above with existing norms in order to offer an overall assessment of the patient's potential tendency towards a biased pattern of responding and the potential validity or lack of validity of the protocol is accomplished by comparing their scores to T-scores for each item.

18. The method of claim 10 wherein the statistical comparison of a patient's pattern of giving potentially biased responses of the kinds described above with existing norms in order to offer an overall assessment of the patient's potential tendency towards a biased pattern of responding and the potential validity or lack of validity of the protocol is accomplished by comparing their scores to the mean values returned on the items by the total sample pool.

19. The method of claim 10 wherein the statistical comparison of a patient's pattern of giving potentially biased responses of the kinds described above with existing norms in order to offer an overall assessment of the patient's potential tendency towards a biased pattern of responding and the potential validity or lack of validity of the protocol is accomplished by analyzing their scores by means of a computer-implemented processor having been configured to use artificial intelligence (including but not limited to: deep learning, neural network modeling, parallel distributed processing, low-rank matrix factorization, regression analysis, vectorization, and skip thought vectors) to analyze patterns in patient data to suggest whether the patient's pattern of responding is likely to compromise the validity of the protocol or not.
